(19) United States
(12) Patent Application Publication
(10) Pub. No.: US 2003/0073125 A1
(43) Pub. Date: Apr. 17, 2003

(54) Methods, systems and software for gene expression data analysis

(75) Inventor: Earl A. Hubbell, Mountain View, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/273,233

(22) Filed: Oct. 16, 2002

Related U.S. Application Data
(60) Provisional application No. 60/329,953, filed on Oct. 16, 2001.

(51) Int. Cl. C12Q 1/68; G06F 19/00; G01N 33/48; G01N 33/50

(52) U.S. Cl. 435/6; 702/20

(57) ABSTRACT

Methods, computer software and systems are provided for biological data analysis. In one embodiment, a probe logarithmic intensity error resolver is provided to analyze gene expression data obtained using multiprobes.

### BACKGROUND OF THE INVENTION

This application claims the priority of U.S. Provisional Application Serial No. 60/329,953, filed on Oct. 16, 2001. The '953 application is incorporated herein by reference for all purposes.

This invention is related to bioinformatics and biological data analysis and visualization.

Many biological functions are carried out by regulating the expression levels of various genes, either through changes in the copy number of the genetic DNA, through changes in levels of transcription (e.g. through control of initiation, provision of RNA precursors, RNA processing, etc.) of particular genes, or through changes in protein synthesis. For example, control of the cell cycle and cell differentiation, as well as diseases, are characterized by the variations in the transcription levels of a group of genes.

Recently, massive parallel gene expression monitoring methods have been developed to monitor the expression of a large number of genes using nucleic acid array technology which was described in detail in, for example, U.S. Pat. No. 5,871,928; de Saizieu, et al., 1998, Bacteria Transcript Imaging by Hybridization of total RNA to Oligonucleotide Arrays, NATURE BIOTECHNOLOGY, 16:45-48; Wodicka et al., 1997, Genome-Side Expression Monitoring in Saccharomyces cerevisiae, NATURE BIOTECHNOLOGY 15:1359-1367; Lockhart et al., 1996, Expression Monitoring by Hybridization to High Density Oligonucleotide Arrays. NATURE BIOTECHNOLOGY 14:1675-1680; Lander, 1999, Array of Hope, NATURE-GENETICS, 21(suppl.), at 3.

Massive parallel gene expression monitoring experiments generate unprecedented amounts of information. Effective analysis of the large amount of data may lead to the development of new drugs and new diagnostic tools. Therefore, there is a great demand in the art for methods for organizing, accessing and analyzing the vast amount of information collected using massive parallel gene expression monitoring methods.

### SUMMARY OF THE INVENTION

In one aspect of the invention, methods, systems and computer software products are provided for conducting biological analysis. The methods, systems and computer software products are particularly suitable for analyzing gene expression data preferably obtained using microarray technology. However, the methods, systems and computer software products are also suitable for analyzing other types of biological data, such as protein profile data.

The invention, as applied to gene expression data, is to construct approximate likelihood functions relating the observed data (probe intensities) to the latent variable of transcript concentration, and possible other latent variables demanded by the model, and thus create good estimators for transcript concentration, by applying standard techniques for constructing good estimates of such variables. In particular, M-estimators can be used to construct estimates for transcript concentration, and the function optimized can easily take into account several disparate constraints, including computational convenience.

One observes across experiments i=1 . . . n a set of probe intensities PM(i,j), MM(i,j) j=1 . . . m. It is assumed that intensity is approximately linear in concentration, and that each probe has different affinity for target, and some unknown background. For PLIER described above, it is assumed that in the ideal case PM(i,j)−MM(i,j)=r(j)*y(i) where r(j) is an affinity term related to the jth probe and y(i) is the concentration of the target in the ith experiment. However, in the real world, there are errors, and they are applied to the observation of each probe. Therefore, when one observe PM(i,j), the true value is IPM(i,j), and the likelihood of observing PM(i,j) given IPM(i,j) is maximized when they are equal, and decreases as they differ.

In the one formulation of PLIER, the following assumptions were made: the error term is multiplicative, and approximately log-normal, and therefore the negative log-likelihood is approximately (log(PM)−log(IPM))ˆ 2+(log(MM)−log(IMM))ˆ 2 for a probe pair, where IPM−IMM=r*y.

Under the further assumption that errors are evenly distributed between PM and MM, this model simplifies with the assumption that (log(PM)−log(IPM))ˆ 2=(log(MM)−log(IMM))ˆ 2. One can further conclude that log(PM)−log(IPM)=log(IMM)−log(MM) yields the maximum log-likelihood (under the balanced error assumption), and deduce therefore that the residual d=abs(log(PM)−log(IPM))=abs(log ((r*y+sqrt((r*y)ˆ 2+4*pm*mm))/(2*pm))) by solving the resulting quadratic equation. This allows one, for a given set of r(j) and y(i) and observed values PM and MM to compute directly an approximate log-likelihood:

LL=−1*sum (i,j) [log (r(j)*y(i)+sqrt((r(j)*y(i))ˆ 2+4*pm*mm))/(2*pm))]ˆ 2.

Choosing r(j) and y(i) to maximize this log-likelihood produces a set of expression estimates y(i) for each experiment that “best” explains the data (requires minimal errors to have happened). This log-likelihood can be made somewhat resistant to error by replacing the squared residual dˆ 2 by a Huber weighting −H(d)=½dˆ 2 for dˆ 2<cˆ 2 and H=abs(d)*c −0.5*cˆ 2 for dˆ 2>cˆ 2. This causes outliers to be somewhat discounted when fitting data. Other robust/resistant weightings of residuals such as biweight estimates can be used.

In summary, Given r(j) and y(i) and observed PM(i,j), MM(i,j), one has a log-likelihood

LL=−1*sum(i,j) F(r(j), y(i), PM(i,j), MM(i,j)) which one can maximize over possible values of r(j) and y(i).

Since concentration is always non-negative, and affinities are always nonnegative, the possible values of these variables are r(j)>=0 and y(i)>=0.

In another aspect of the invention, various extensions and applications are provided.

### DETAILED DESCRIPTION

Reference will now be made in detail to the exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. No. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent applications 10/063,559, 60/349,546, 60/376,003, 60/394,574, 60/403,381.

Definitions

Nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. “Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

Hybridization conditions will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point ™ fro the specific sequence at s defined ionic strength and pH. The Tm is the temperature (under defined ionic strength, pH and nucleic acid composition) at which 50% of the probes complementary to the target sequence hybridize to the target sequence at equilibrium. Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2^{nd} Ed. Cold Spring Harbor Press (1989) and Anderson “Nucleic Acid Hybridization” 1^{st} Ed., BIOS Scientific Publishers Limited (1999), which are hereby incorporated by reference in its entirety for all purposes above.

Hybridization probes are nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501 filed Apr. 3, 1996.

Probe: A probe is a molecule that can be recognized by a particular target. In some embodiments, a probe can be surface immobilized. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A fragment, segment, or DNA segment refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3^{rd} Ed. Cold Spring Harbor Laboratory Press, Cold Spring Harbor, New York (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

I. Gene Expression Monitoring using Microarrays

High density nucleic acid probe arrays, also referred to as “DNA Microarrays,” have become a method of choice for monitoring the expression of a large number of genes. Typically, a nucleic acid sample is a labeled with a signal moiety, such as a fluorescent label. One of skill in the art would appreciate that, while labeling is preferred in some embodiments, it is not required. Detection methods without labeling are also available.

The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine florescence intensities distribution. Typically, the hybridization of each probe is reflected by several pixel intensities. The raw intensity data may be stored in a gray scale pixel intensity file. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells. The probes in a cell are designed to have the same sequence (i.e., each cell is a probe area). A CEL file may contain the statistics of a cell, e.g., the 75 percentile and standard deviation of intensities of pixels in a cell. The 75 percentile of pixel intensity of a cell is often used as the intensity of the cell. Methods for signal detection and processing of intensity data are additionally disclosed in, for example, U.S. Pat. Nos. 5,547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996, 6,136,269 and attorney docket numbers 3298.1 and 3309, all of which are incorporated by reference in their entireties for all purposes.

The embodiments of the invention will be described using GeneChip® high oligonucleotide density probe arrays (available from Affymetrix, Inc., Santa Clara, Calif., USA) as exemplary embodiments. One of skill the art would appreciate that the embodiments of the invention are not limited to high density oligonucleotide probe arrays. In contrast, the embodiments of the invention are useful for analyzing any parallel large scale biological analysis, such as those using nucleic acid probe array, protein arrays, etc.

Gene expression monitoring using GeneChip® high density oligonucleotide probe arrays are described in, for example, Lockhart et al., 1996, Expression Monitoring By Hybridization to High Density Oligonucleotide Arrays, Nature Biotechnology 14:1675-1680; U.S. Pat. Nos. 6,040,138 and 5,800,992, all incorporated herein by reference in their entireties for all purposes.

In the preferred embodiment, oligonucleotide probes are synthesized directly on the surface of the array using photolithography and combinatorial chemistry as disclosed in several patents previous incorporated by reference. In such embodiments, a single square-shaped feature on an array contains one type of probe. Probes are selected to be specific against desired target.

In a preferred embodiment, oligonucleotide probes in the high density array are selected to bind specifically to the nucleic acid target to which they are directed with minimal non-specific binding or cross-hybridization under the particular hybridization conditions utilized. Because the high density arrays of this invention can contain in excess of 1,000,000 different probes, it is possible to provide every probe of a characteristic length that binds to a particular nucleic acid sequence.

Probes as short as 15, 20, 25 or 30 nucleotides are sufficient to hybridize to a subsequence of a gene and that, for most genes, there is a set of probes that performs well across a wide range of target nucleic acid concentrations. In a preferred embodiment, it is desirable to choose a preferred or “optimum” subset of probes for each gene before synthesizing the high density array.

In some preferred embodiments, the expression of a particular transcript may be detected by a plurality of probes, typically, up to 5, 10, 15, 20, 30 or 40 probes. Each of the probes may target different sub-regions of the transcript. However, probes may overlap over targeted regions.

In some preferred embodiments, each target sub-region is detected using two probes: a perfect match (PM) probe that is designed to be completely complementary to a reference or target sequence. In some other embodiments, a PM probe may be substantially complementary to the reference sequence. A mismatch (MM) probe is a probe that is designed to be complementary to a reference sequence except for some mismatches that may significantly affect the hybridization between the probe and its target sequence. In preferred embodiments, MM probes are designed to be complementary to a reference sequence except for a homomeric base mismatch at the central (e.g., 13th in a 25 base probe) position. A probe pair is usually composed of a PM and its corresponding MM probe. The difference between PM and MM provides an intensity difference in a probe pair.

II. Probe Logarithmic Intensity Error Resolver (PLIER)

In one aspect of the invention, a simplified expression analysis estimator base is provided. The underlying model has two assumptions. One of skill in the art would appreciate that the assumptions are used for establishing the models. The utility of the methods, computer software and computer systems may not be necessarily constrained by the assumptions. First, probe intensities are assumed to have logarithmic errors arising from either measurement or expression fluctuation. Second, it is assumed that the likelihood of the data can be approximated by some reasonable function of the log-scale errors. That is, every error is a multiplicative factor. It is desirable to minimize the error across all the probes.

The model for intensity and error is:

PM(j)=(b(j)+A(j)*C(i))*a(j)

MM(j)=(b(j)+A′(j)*C(i))*a′(j)

where log(a(j)), log(a′(j)) are approximately normally distributed error terms.

All PM-MM pairs (with the same affinity) should give identical values for a given expression level, y. Assuming that multiplicative errors occur implies x=aPM−bMM for all probe pairs, and errors a(j), a′(j) in each probe pair. It is desired to minimize some function of log(a), log(a′) to maximize the likelihood. For example, one could use the sum of log(a)ˆ 2+log(a′)ˆ 2 over all probe pairs, which is a standard least-squares answer. While the analytic minimum is complicated, the assumption of balanced error between PM and MM, allows for a nice simplification, since this implies (for each pair) that log(a)=+−log(b).

Keeping the physical constraints x>=0, a>0, PM>0, MM>0

One can then directly derive:

a=(x/(PM−MM)) for log(a)=log(b), PM>MM, x>0.

a=(1/(2*PM))*(x+sqrt(x*x+4*PM*MM)) for log(a)=−log(b).

Note that only positive values are used in this calculation, but, in some embodiments, x can be allowed to equal zero. Further note that in all cases, the second residual value is of smaller absolute log-value than the first residual value, so we may always simply compute using the second formula.

So for any value of x, a quantity proportional to the log-likelihood can be calculated rapidly. This allows for an approximate maximum likelihood approximation for x by either gradient search or evaluation of evenly spaced plausible values for x (e.g., 0-65,536).

In some embodiments, this approach is extended to a multiple array analysis by allowing a multiplicative constant r(j) unique to each pair of probes to enter our analysis. This constant can be a part of the “error” term in the single-array case, but can be fit across multiple arrays (“probe-effect”). That is, every PM−MM pair should give the value x(i,j)=r(j)*y(i) for a given probe pair j in experiment (i), except for multiplicative errors.

In this case, r(j)*y(i) is substituted in the preceding derivation and obtain

(i,j)=(1/(2*PM(i,j)))*(r(j)*y(i)+sqrt(y(i)*y(i)+4*PM(i,j)*MM(ij)))

for the ith experiment and the jth probe pair.

Now, one can optimize across all y(i) and r(j) (constraining, e.g., the product of r(j) to be 1.0 to allow for identifiability).

Note that when y=0, a nice relationship (log(a)ˆ 2+log(a′)ˆ 2)=(log(PM)−log(MM))ˆ 2 is obtained.

This analysis may be made robust against outliers by optimizing the median sum of squares, or other such methods of rejecting outliers by repeatedly fitting information. Since residual error terms are explicitly produced, one can obtain confidence intervals by bootstrapping. One can also approximate the likelihood surface because of the particularly easy form of the residuals, which provides another method of estimating variation.

In another aspect of the invention, there is a rapid approximation to PLIER using a weighted least-squares fit. The assumption of multiplicative errors on the observed intensity leads directly to the inference that not all PM-MM differences are equally variable: PM−MM differences with “small” PM and MM values are less variable than the same difference with “large” PM and MM values. If a fixed level of multiplicative error is assumed, it is expected that the variance of an intensity value is proportional to the intensity squared. That is, a given PM value has variance c*PMˆ 2, for some constant c. Combining the two, a given PM−MM difference is expected to have variance approximately c*(PMˆ 2+MMˆ 2). Making the assumption that the resulting distribution is near-normal, one can approximate an appropriate weighting on the residuals by weighting every PM−MM difference by 1/(PMˆ 2+MMˆ 2).

That is, e=(PM−MM-r*y), where e is the residual, and is approximately distributed as N(0, 1/c*(PMˆ 2+MMˆ 2)). Since c is constant across all observed intensities (by assumption), one can fit to the minimum of LL=sum(eˆ 2/(PMˆ 2+MMˆ 2)) and find an approximation to the value one would get from the multiplicative error. One advantage to this formulation is that the optimum value for any affinity r or expression level y can be found by simple differentiation, and solved in an explicit formula:

y(i)=sum (r(j)*(PM(i,j)−MM(i,j))/(PM(i,j)ˆ 2+MM(i,j)ˆ 2))/sum(r(j)*r(j)/(PM(i,j)ˆ 2+MM(i,j)ˆ 2) (sum over j) and

r(j)=sum(y(i)*(PM(i,j)−MM(i,j))/(PM(i,j)ˆ 2+MM(i,j)ˆ 2)/sum(y(i)*y(i)/(PM(i,j)ˆ 2+MM(i,j)ˆ 2) (sum over i).

These formulae can be used to “polish” the table to a solution for expression values and affinities.

An extension of these formulae to smooth the probe affinities towards 1.0 (avoiding undue weight on any single probe) is to add a penalty term to both the top and the bottom of the r(j) formula, thereby smoothing the resulting value.

A further extension of this approach is to instead take either least-absolute residual or least-Huber residual fits, both of which have tractable derivatives. However, both these fits depend on the signs of the residuals, and therefore need slightly more computational power for the search (but still not as much as for log-scaled residuals).

These fits can also be extended to the “full model”: PM(ij)=r(j)*y(i)+v(j)*y(i)+b(j) and MM(i,j)=v(j)*y(i)+b(j), where the background b(j) is assumed to be constant across all experiments. Similar explicit formulae for finding v(j) and b(j) assuming multiplicative errors approximated by arithmetic errors with variance proportional to intensity squared can be produced.

III. Additional Embodiments

In some embodiments, one feature of the PLIER was to assign multiplicative errors to the intensity of individual probes, rather than assigning a fixed error to the difference. This allowed for a rapid approximation of a log-likelihood and the construction of an M-estimator for expression level and probe effects across multiple chips. In another aspect of the invention, several useful extensions and alternate M-estimators are provided.

One observes across experiments i=1 . . . n a set of probe intensities PM(i,j), MM(i,j) j=1 . . .m. It is assumed that intensity is approximately linear in concentration, and that each probe has different affinity for target, and some unknown background. For PLIER described above, it is assumed that in the ideal case PM(i,j)−MM(i,j)=r(j)*y(i) where r(j) is an affinity term related to the jth probe and y(i) is the concentration of the target in the ith experiment. However, in the real world, there are errors, and they are applied to the observation of each probe. Therefore, when one observe PM(i,j), the true value is IPM(i,j), and the likelihood of observing PM(i,j) given IPM(i,j) is maximized when they are equal, and decreases as they differ.

In the one formulation of PLIER, the following assumptions were made: the error term is multiplicative, and approximately log-normal, and therefore the negative log-likelihood is approximately (log(PM)−log(IPM))ˆ 2+(log(MM)−log(IMM))ˆ 2 for a probe pair, where IPM−IMM=r*y.

Under the further assumption that errors are evenly distributed between PM and MM, this model simplifies with the assumption that (log(PM)−log(IPM))ˆ 2=(log(MM)−log(IMM))ˆ 2. One can further conclude that log(PM)−log(IPM)=log(IMM)−log(MM) yields the maximum log-likelihood, and deduce therefore that the residual d=abs(log(PM)−log(IPM))=abs(log ((r*y+sqrt((r*y)ˆ 2+4*pm*mm))/(2*pm))) by solving the resulting quadratic equation. This allows one, for a given set of r(j) and y(i) and observed values PM and MM to compute directly an approximate log-likelihood:

LL=−1*sum (i,j) [log (r(j)*y(i)+sqrt((r(j)*y(i))ˆ 2+4*pm*mm))/(2*pm))]ˆ 2.

Choosing r(j) and y(i) to maximize this log-likelihood produces a set of expression estimates y(i) for each experiment that “best” explains the data (requires minimal errors to have happened). This log-likelihood can be made somewhat resistant to error by replacing the squared residual dˆ 2 by a Huber weighting −H(d)=½dˆ 2 for dˆ 2<cˆ 2 and H=abs(d)*c −0.5*cˆ 2 for dˆ 2>cˆ 2. This causes outliers to be somewhat discounted when fitting data.

In summary, Given r(j) and y(i) and observed PM(i,j), MM(i,j), one has a log-likelihood

LL=−1*sum(i,j) F(r(j), y(i), PM(i,j), MM(i,j)) which one can maximize over possible values of r(j) and y(i).

r(j)>=0 and y(i)>=0.

Extension: Use expected value

These estimates are approximate maximum likelihood estimates. Instead of estimating an expression level as the value y(i), one can instead estimate the expression level by E[y(i)], the expected value of y(i) given the likelihood surface. One compute this by numerical integration of possible values of y in the ith experiment coupled with their likelihood. That is, the weighted sum ey(i)=sum(x) x*exp(LL(x)/LL(y(i))/sum(x) (exp(LL(x)/LL(y(i)) is computed, where LL(y(i)) is a crude estimate of the variance. More sophisticated approximations to the expected value are also possible.

This value has two advantages: first, this is an estimate that theoretically minimizes the squared error between the true value and the estimate, and second, it is always positive. It has the noticeable drawback that it involves more computation and slightly more assumptions about the distribution of errors.

Extension: Replicate analysis

Replicate analysis is also provided in PLIER. Simply set a single concentration y to be fit across all replicate experiments within a group (that is, one concentration per replicate group, as opposed to one concentration per experiment). Maximizing the likelihood is done as above.

Extension: Bootstrap

Bootstrapping PLIER can be done in several ways. When using replicate analysis, bootstrapping can be performed by resampling at the experiment level. It is also possible to resample at the residual level. For every probe pair, one can implicitly computed IPM and IMM the “true values”. Keeping the “true values” fixed, one can resample the paired errors log(IPM)−log(PM), log(IMM)−log(MM) and assign a new set of “observed values” by assigning new errors to the IPM,IMM values. One can then refit the likelihood to find new concentration estimates y. Bootstrapping allows for confidence intervals to be computed based on either biological variability (experiment level) or technical variation (error resampling).

Extension: Poisson distribution error

Instead of obtaining an approximate log-likelihood from assuming a log-normal distribution, one can assume the observations come from a different distribution. For example, a Poisson distribution is typically used to represent count data, and has the likelihood f(x)=(lambda)ˆ (−x)eˆ (−lambda)/(x!) for x a non-negative integer, and lambda is an estimate of the “rate”. Since one observes “counts” of photons from the label, “lambda” is proportional to the number of molecules absorbed, and by the usual assumption, is related to the affinity and concentration (plus background). One then has lambda(PM)−lambda(MM)=r*y. The log-likelihood can then be approximated by:

LL=pm*log(lambda(PM))−(lambda(PM)+mm*log(lambda(MM)−(lambda(MM))−PM*log(PM)−MM*log(MM)

and applying our relation lambda(PM)−lambda(MM)=r*y, and solving the resulting quadratic:

avg=(pm+mm)/2;

lambda(MM)=0.5 *(avg-r*y+sqrt((avg-r*y)*(avg-r*y)+2*mm*r*y));

−LL=pm*log(r*y+lambda)−(lambda+r*y)+mm*log(lambda)−lambda−pm*log(pm)−mm*log(mm);

One can then optimize in r and y as above to obtain expression estimates. This approximation serves for the case that the variance increases linearly with intensity (log-scale has variance increasing as the square of intensity). As a side effect, the appropriate “variance stabilizing” transformation is z=sqrt(y).

Extension: Priors on probe affinities

A common problem with M-estimators can be seen as well with PLIER. Finding the maximum of a distance function on data uncertain in two axes (concentration and affinity in this case) is quite vulnerable to outlying points. A typical example has zero (true) concentration across all experiments, and a single outlier in one experiment. A well-fitting solution assigns near-zero affinity to all probes but the outlier, and zero concentration to all experiments but the one containing the outlier. This is typically an unacceptable solution. One solution is to replace the sum of all the error metric with a median.

Another solution is to apply a Bayesian prior to the probe affinities. That is, one can assume that the affinities come from a log-normal distribution with some variance (specified or fit to the experiment), and therefore provide a penalty term to the log-likelihood for probe affinities far from 1.0. That is, one can add a term q*(log(r))ˆ 2, where q is a term relating to the ratio of the variance of probe affinities and the variance of error terms. In practice, this is simply a specifiable parameter in the optimization. This penalizes any solutions with unusually great affinity spread between probes. As above, one can use a Huber penalty instead.

This has the additional advantage of allowing “tuning” between single-experiment and multiple experiment analyses—if q is taken to be extremely large, the probe affinities will be fixed at 1.0, just as the single-experiment assumption requires, while if q is extremely small, this defaults to the ordinary multiple-experiment fit.

Extension: Priors on concentration

Similarly, priors can be put on the concentration values. Under a log-normal prior, again this takes the shape of a penalty term s*(log(c)−log(z))ˆ 2, where z is the most likely concentration for a randomly chosen transcript and s is again a tuning parameter relating to the ratio of variances.

A more complicated prior involves the “shrinkage” assumption that typically concentrations do not vary from experiment to experiment for most transcripts. This provides a penalty term of the form s*(log(c)−log(c′))ˆ 2 for all pairs of concentrations c and c′.

Note that concentration must be positive for each of these assumptions.

A similar approach was suggested in RECOMB 2002 “BEAM” for analyzing data, however, additional additive error terms were thought necessary, and “BEAM” did not analyze a full model fit to the probes.

Extension: Use of global background

It has been suggested by several authors (Speed, Irizarry, Naef) that a “global background” value be used instead of a background unique to each probe. It is typically assumed that the background value b is measured to a very fine precision, and therefore can be treated as a constant. However, any individual probe can still be subject to measurement error, yielding intensity values “below background”. Simple subtraction of this background causes negative values for such probes.

The PLIER formulation works equally well with a global background value. As before, it is assumed that there is a multiplicative error on the measured intensity of the perfect match probe, and assume the following is true: IPM-b=r*y, where b is the global background, r is the affinity, and y is the concentration. Note that this completely specifies the value of IPM=r*y+b. The required error term is then d=(log((r*y+b)/pm)). As above, one can either square this error term, or use the huber formulation.

One can then find r, y as our approximate maximum likelihood estimates.

Extension: Approximate global background computation—expected value subtraction

A popular method for background “subtraction” is to assume a distribution for this background and take the positive expected value of the result, given an observation. This is typically done with a moderately complicated normal approximation. The typical formula is integral (x*p(x)dx (x>0)/integral(p(x)dx) (x>0)), where x is the amount above background (The observed intensity enters into the calculation of p(x)).

The complicated integral can be simplified drastically by assuming a simpler distribution underlying the background. Two such simple distributions are uniform and triangular distributions over the “background” range.

Let background be represented by a uniform distribution running from the least observed intensity Low, to 2*b-Low where b is the global background value. Then the expected value EPM=PM−B if PM>2*b-Low, and (PM-Low)/2 otherwise. (To maintain positivity, a small value may be added to these quantities if desired.)

Let background be represented by a triangular distribution running from the least observed intensity Low to 2*b-Low, where b is the global background value, and the most likely value for a probe at background. There are three cases here: EPM=PM−B if PM>2*b-Low, EPM=(PM−B)/3+(PM-Low)/3 if PM>B, EPM=(PM-Low)/3 otherwise.

Any relatively simple distribution represented by piecewise polynomial pieces yields a simple formula for this expected value. Additional distributions such as the one induced by the biweight kernel (1-ˆ 2)ˆ 2 are also possible simplified approximations.

Such approximations provide values relatively close to the values provided by normal theory (which itself is an approximation to the underlying data) and so serve essentially as well as the normal theory background adjustment.

This method of background adjustment is used as a “preprocessing” term on intensity, after which the resulting intensity is used in a model fit (PLIER or others). For PLIER, the error term is then the trivial multiplicative error d=abs(log(r*y/pm)), where pm is the adjusted perfect match intensity, and fit to r and y are done as usual.

Extension: Binding fit to intensity

A common failing of standard models for expression is that the dependence of intensity upon concentration is not linear. One common cause for nonlinearity is that binding sites on the surface saturate and thus there is a maximum value which the observed probe intensity approaches as concentration increases. In particular, one such model is r*y=(I−b)/(X−I) where r*y is affinity times concentration, I is the observed intensity, b is the background, and X is the maximum value possible. By substituting the true intensity a*I for the observed intensity (a a multiplicative error), given r,y, X, and b and an observed intensity I, a=(r*y*X+b)/(I*(1+r*y)). As usual, one can find an optimum set of r,y,X,b values for each probe set across experiments by minimizing the residuals ln(a). This accounts for some first-order saturation behavior, and therefore allows concentration to be more accurately estimated at high concentrations (as well as better accounting for the variation).

Note that the assumption of error on the intensity simplifies the process of constructing a residual, even with a more complicated function linking intensity to concentration.

Additional applications for approximate likelihood techniques include genotyping, variant detection, and alternate splicing analysis. Each of these problems can be formulated in a maximum likelihood manner, and approximated to form an M-estimator.

In one application of the invention to genotyping, multiplicative errors are assumed for probe intensity measurements, and a linear model for intensity for a given concentration is assumed, however, extra terms are added to the likelihood to account for the two different transcripts possibly present in an SNP. The intensity of a given probe (whether PM or MM) is given by I=ra*ca+rb*cb+B, where ra, rb are the affinities of the probe to the A form of the transcript and B form of the transcript respectively, and ca, cb are the concentrations of the A form of the transcript and the B form of the transcript, and B is a background for that probe. For the particular case of samples taken from diploid individuals, we can restrict the concentrations to the following four cases ca=cb=0 (no amplification), ca=0 (homozygous BB),cb=0 (homozygous AA), ca=cb>0 (heterozygous AB). A multiplicative error is then assigned to each intensity to make the equation e*I=ra*ca+rb*cb+B exactly true for all probes. The latent variables ra, rb, ca, cb, B can then be fitted over all experiments and probes as above to minimize the resulting approximate likelihood.

In another aspect of the invention, methods for analyzing alternative splicing data are provided. In one embodiment, there may be multiple transcripts that may hybridize to each probe, and (for known splice forms) equations I=sum(r*c)+B can be constructed where the sum is over all possible splice forms hybridizing to a given probe. As above, one can maximize the likelihood obtained from the multiplicative errors to find the latent variables describing the concentration for each splice variant. Further, unknown splice variants can be detected by unusually large residuals in the fit.

Finally, the same description illustrates the formulation of the invention for variant detection. The intensity of each probe is a sum of affinities for possible transcripts [with single base errors] times the concentration of such transcripts. Given the four possible substitutions, there are four affinity terms and four possible concentrations. Assuming diploid individuals, there is the constraint that at most two of those concentrations are non-negative. By maximizing the likelihood over all possibilities, one can find the latent variables indicating which sequence variants are present. The fit may be improved by including terms representing probes overlapping the variant point [‘footprints’] and including the decreased affinity expected to sequence variants explicitly in the intensity. Such additional terms are likely to increase the sensitivity and accuracy of the estimation.

In addition, the explicit representation of affinity terms allows for the possibility of deconvolving gene families, in which significant cross-hybridization is predicted to occur among members of the family

The present inventions provide methods and computer software products for analyzing biological data. It is to be understood that the above description is intended to be illustrative and not restrictive. Many variations of the invention will be apparent to those of skill in the art upon reviewing the above description. The scope of the invention should, therefore, be determined not with reference to the above description, but should instead be determined with reference to the appended claims, along with the full scope of equivalents to which such claims are entitled.

All cited references, including patent and non-patent literature, are incorporated herewith by reference in their entireties for all purposes.

What is claimed is:

1. A method for analyzing gene expression data comprising:
Providing perfect match intensity (PM) and mismatch intensity (MM) for a plurality of probes (j) against a target nucleic acid in a number of experiments (i);
Maximizing the likelihood: LL=−1*sum(i,j)F(r(j), y(i), PM(I,j), MM(i,j)) to obtain a value for r(j) and y(i), where ro) is an affinity term related to the jth probe and y(i) is the concentration of the target in the ith experiment and r(j)>=0 and y(i)>=0.

2. The method of claim 1 wherein LL=−1* sum(i,j) [log(log(r(j)*y(i)+sqrt((r(j)*y(i)ˆ 2+4*PM*MM))/(2*PM))]ˆ 2.

3. A method for analyzing gene expression data comprising:
Providing the intensities for a plurality of probes (n) against a target nucleic acid in a number of experiments (m);
Providing an approximate likelihood function LL=sum (i,j) F(r(j), y(i), I(i,j)), where F depends on r(j) a plurality of probe specific parameters, and y(i) is the concentration of the target in the ith experiment
Estimating y(i) by maximizing the likelihood function.

4. A method for analyzing gene expression data comprising:
Providing the intensities for a plurality of probes (n) against a target nucleic acid in a number of experiments (m);
Providing an approximate likelihood function LL=sum (i,j) F(r(j), y(i), I(i,j)), where F depends on r(j) a plurality of probe specific parameters, and y(i) is the concentration of the target in the ith experiment; and
Estimating y(i) by polishing the likelihood function.

5. A method for analyzing gene expression data comprising:
Providing the intensities for a plurality of probes (n) against a target nucleic acid in a number of experiments (m);
Providing an approximate likelihood function LL=sum (i,j) F(r(j), y(i), I(i,j)), where F depends on ro) a plurality of probe specific parameters, and y(i) is the concentration of the target in the ith experiment; and
Estimating y(i) by the expected value obtained from the likelihood surface from this likelihood function.

6. A method for analyzing gene expression data comprising:
Providing the intensities for a plurality of probes (n) against a target nucleic acid in a number of experiments (m);
Providing an approximate likelihood function LL=sum (i,j) F(r(j), y(i), I(i,j))+sum(q(r(j)))+sum(z(y(i)), where F depends on r(j) a plurality of probe specific parameters, and y(i) is the concentration of the target in the ith experiment, and q is a penalty function depending on the probe specific parameters and and z is a penalty function depending on concentration; and
Estimating y(i) by maximizing this likelihood function.
